(19) United States
(12) Patent Application Publication
(10) Pub. No.: US 2008/0319419 A1
(43) Pub. Date: Dec. 25, 2008

(54) Medical catheter and a catheter assemble

(75) Inventors: Tomihisa Kato, Aichi-ken (JP); Fuminobu Yoshimachi, Aomori-Ken (JP)

Correspondence Address: MORGAN LEWIS & BOCKIUS LLP, 1111 PENNSYLVANIA AVENUE NW, WASHINGTON, DC, 20004, US

(73) Assignees: Asahi Intecc Co., Ltd., Aichi-ken (JP); Filmecc Co., Ltd., Aichi-ken (JP)

(21) Appl. No.: 12/153,275

(22) Filed: May 15, 2008

(30) Foreign Application Priority Data
May 31, 2007 (JP) 2007-146233

(51) Int. Cl. A61M 25/09

(52) U.S. Cl. 604/528

(57) ABSTRACT

In a medical catheter and a catheter assemble, a front opening 6a of a second lumen 6 is provided on a catheter 1. The front opening 6a acts as a lateral slope opening 8 inclined upward from a distal end T to a proximal end S along a common wall 7 of a dual lumen (5, 6). A second guide wire 10 extends its distal end 10a beyond the lateral slope opening 8. By operating the distal end 10a along a peripheral edge 8A of the lateral slope opening 8, it is possible to project a sphere in a three-dimensional perspective in which the distal end 10a can operationally move freely so as to extend a detectable region for a perforative hole against minute blood stream paths (n) developed in an occluded area of the blood vessel N.

The present invention claims priority to Japanese Patent Application No. 2007-146233 filed May 31, 2007, the disclosure of which is incorporated herein by reference in its entirety.

### BACKGROUND OF THE INVENTION

1. Field of the Invention

The invention relates to a medical catheter having a dual lumen, through which the corresponding guide wires are inserted, and particularly concerns to a medical catheter and catheter assemble which enables an operator to freely shift a distal end of the guide wires so as to extend a detectable region for a perforative hole against minute blood stream paths in an occluded portion of the blood vessel.

2. Description of Prior Art

In a medical catheter in which guide wires are inserted into two lumens laterally arranged side by side, a distal end portion is slantwisely severed against an axial direction to form a V-shaped sphenoid structure with a partition wall between the two lumens placed in front as shown by Japanese Laid-open Patent Application No. 2006-223338.

The two lumens has corresponding first and second front openings formed to be a semi-elliptical in shape so as to enable the operator to advance the guide wires extensively through the first and second front openings.

In the above catheter, upon operating the guide wire forward through the lumens, each distal end of the guide wires are adapted to move toward a bifurcated portion of the blood vessel. When each distal end of the guide wires reach the bifurcated portion, the front openings easily enables an operator to selectively advance one of the guide wires into one of the bifurcated paths of the blood vessel.

In a triple lumen catheter used to smoothly implement the blood infusion and blood depletion at the time of undergoing the dialysis as shown by Japanese Laid-open Patent Application No. 2006-95134, the catheter has a distal end severed slantwise to form an infusion opening at a cone-shaped leading end portion. An open end structure keeps the infusion opening in good condition so as to avoid a shortage of the blood transfer.

In a dual wire catheter which is used to set a mesh-work tube in a bifurcated portion of the abdominal aorta as shown by Japanese Published Patent Application No. 2003-504127, a first wire is placed between the heterolateral iliac artery and the ortholateral iliac artery, and a second wire passes a part of the ortholateral iliac artery to extend into the aorta, so as to insure a therapeutical improvement against the abdominal aortic aneurysm with an efficient introductory operation of a dual tube-lumen catheter.

In the medical catheter (Japanese Laid-open Patent Application No. 2006-223338), the guide wires have the respective distal end extensions operationally movable within the first and second openings, so as to easily enable the operator to selectively advance the guide wires into the bifurcated paths of the blood vessel when navigating the guide wires back and forth through the respective lumens after setting the catheter in the somatic cavity (blood vessel).

Such is the structure that a movable region permitted for the distal end extensions to shift, is confined to a two-dimensional area within a plane generally perpendicular to the axial direction along a width of the minor diameter of the openings because of the openings being semi-elliptical in shape.

For this reason, it is not appropriate to adopt the above structure as a catheter used for detecting perforative holes against the occluded area of the blood vessel.

It has been desired to introduce a medical catheter which enables the users to extend the detectable region for the perforative holes against the vascular occlusion area, so as to attain a therapeutical improvement in a significant degree against the diseased area.

It is often that the vascular occlusion area is not completely obturated with minute blood stream paths developed through the occlusion area. It is a very effective way to detect the minute blood stream path, so that the guide wire can introduce its distal end into the minute blood stream path to enlarge the path as a perforative hole. Upon detecting the minute blood path, the operator takes one guide wire, and puts its distal end against the occlusion area, while at the same time, the operator takes the other guide wire to make its distal end reciprocally move forward and rearward in a three-dimensional way including rotational manipulation.

The catheter is generally not only thin but long in the axial direction, and the front openings are semi-circular, a planar open surface of which is generally perpendicular to the axial direction of the catheter. Otherwise, the front openings are semi-elliptical, a planar open surface of which is oblique against the axial direction. Because of the long and thin guide wire, it is by no means easy to make the proximal end follow the distal end of the guide wire upon operationally transmitting the former rotational movement to the latter.

Because of the front openings being semi-circular or semi-elliptical, the movable region is restricted to a detectable area permitted for the respective distal ends to move within the front openings. For this reason, there has been no technological idea to make the guide wires detect minute blood stream paths to perforate the occluded area of the blood vessel. This holds true with Japanese Laid-open Patent Application No. 2006-95134 and Japanese Published Patent Application No. 2003-504127.

Therefore, it is an object of the invention to overcome the above drawbacks so as to provide a medical catheter and a catheter assemble in which guide wires extends their distal ends beyond front openings to enable the distal ends to move in three-dimensional way, so as to extend a detectable region for the distal ends to explore a perforative hole against minute blood stream paths, thus making it extremely easy to find the minute blood stream paths upon operationally moving respective guide wires forward and rearward through respective lumens.

### SUMMARY OF THE INVENTION

According to the present invention, there is provided a medical catheter which has a dual lumen each partitioned by a common wall. A distal end of the dual lumen has an opening area, through which a guide wire is introduced to pass. At least one of the opening areas of the dual lumen inclines as a lateral slope opening along the common wall from a distal portion to a proximal portion of the dual lumen.

With the distal end of the guide wire extended beyond the lateral slope opening, it is possible for the distal end to direct the movable or shiftable region in a predetermined three-dimensional way, thus extending a detectable region for the distal ends to explore a perforative hole against minute blood stream paths so as to make it remarkably easy to operationally fine the minute blood stream paths.

According to the other aspect of the present invention, a catheter body has a circular cross section, and each of the dual lumen has a D-shaped cross section and the same outer circumferential length.

With the lumens having the D-shaped cross section and the catheter body having the circular cross section, it is possible to insure a good insertability of the guide wires against the lumens so as to resultantly improve a maneuverability upon operating the guide wires.

According to the other aspect of the present invention, one of the dual lumen has an outer circumferential length greater than that of the rest of the dual lumen. The lateral slope opening resides on the lumen, an outer circumferential length of which is greater than that of the rest of the dual lumen.

With the lateral slope opening residing on the lumen, an outer circumferential length of which is greater than that of the rest of the dual lumen, it is possible to extensively explore a position suitable for detecting the perforative holes. With small amount of rotational operation of the catheter, it is possible to quickly explore an entire surface of an inner wall of the blood vessel in the circumferential direction so as to insure a significant therapeutical improvement.

According to the other aspect of the present invention, a distal end of the common wall is formed to be arcuately convex-shaped configuration.

Upon exploring the position suitable for detecting the perforative holes, the catheter is operationally rotated while pressing the distal end against the diseased area (occluded area) of the blood vessel. This makes the distal end smoothly engage with the diseased area to render the rotational operation easy so as to facilitate the rotational maneuver.

According to the other aspect of the present invention, the lumen which has the lateral slope opening, has a head portion having a cylindrical slope surface, semi-bullet surface or semi-spherical surface at a distal edge of the lateral slope opening.

Upon exploring the region suitable for detecting the perforative holes, the catheter is operationally rotated while pressing the head portion against the diseased area (occluded area) of the blood vessel. This makes the head portion smoothly engage with the diseased area to render the rotational operation easy, thus facilitating the rotational maneuver so as to insure a less intrusive and quicker therapy against a patient.

According to the other aspect of the present invention, a catheter assemble of the catheter and guide wires inserted into the dual lumen is provided. The distal end portions of the guide wires are in the form of a curved or arcuate configuration and having one, two or three inflection portions with a total bending angle limited within 90 degrees.

This enables the guide wires to reduces their distal ends, thus enabling users to extend the detectable region in the diametrical direction upon so as to explore the perforative hole with a shortened distance traveled in the axial direction.

In particular, the shortened distal end makes it effective to therapeutically treat the diseased area of the iliac artery, an internal diameter of which tends to increase.

According to the other aspect of the present invention, a guiding catheter is provided which has an arcuately bent portion having an angular range of 130-230 degrees at a remote portion away by 50-150 mm from a distal end of the guiding catheter. An elongate sheath is inserted into the arcuately bent portion so as to stretch the arcuately bent portion with the guide wire inserted into the elongate sheath.

Upon inserting a guiding catheter into a bifurcated portion of the iliac artery to therapeutically treat the diseased area of the iliac artery, because the bifurcated portion forms a steep angular portion as a chevron-shaped configuration, it is necessary for an operator to sharply bend a catheter assemble by more than 130 degrees against the direction in which the catheter assemble is inserted. For this reason, it is by no means easy to set a straight tube portion of the guiding catheter in the bifurcated portion of the iliac artery.

When the guiding catheter is subjected to an operational reaction on the way to advance over the bifurcated portion of the iliac artery, the operational reaction would float the guiding catheter (approx. 2.0 mm in dia.) off the bifurcated portion of the iliac artery (approx. 20.0 mm in dia.).

Contrary to the straight tube portion of the above guiding catheter, the guiding catheter, according to the invention, has the arcuately bent portion having the angular range of approx. 130-230 degrees at a proximal portion away by approx. 50-150 mm from a distal end of the guiding catheter. It is possible to insure a sufficient length of the arcuately bent portion at the heterolateral iliac artery, thus preventing the guiding catheter from coming off the bifurcated portion of the iliac artery without floating in the blood streams, so as to attain the therapeutical improvement against the diseased area.

### BRIEF DESCRIPTION OF THE DRAWINGS

Preferred forms of the present invention are illustrated in the accompanying drawings in which:

FIG. 1 is a side elevational view of a rapid-change type catheter assemble according to a first embodiment of the invention;

FIG. 2 is a perspective view of a distal end portion of the medical catheter;

FIG. 3 is a side elevational view of the rapid-change type catheter assemble for use in a diseased area of a blood vessel;

FIG. 4 is a latitudinal cross sectional view taken along the line IV-IV of FIG. 2;

FIGS. 5, 6 are side elevational views of the distal end portion of the medical catheter;

FIG. 7 is a perspective view of an engagement tool;

FIG. 8 is a side elevational view of a comparative catheter assemble for use in the diseased area of the blood vessel;

FIGS. 9-12 are plan views of a distal end portion of a comparative guide wire;

FIGS. 13-14 are side elevational views of the catheter assemble for use in the diseased area of the blood vessel;

FIG. 15 is a schematic view of the medical catheter of the catheter assemble;

FIG. 16 is a schematic view showing the medical catheter rotated somewhat from the position of FIG. 15;

FIG. 17 is a plan view of the distal end of the medical catheter of FIG. 14;

FIG. 18 is a plan view showing a lateral slope opening provided on a distal end of a first lumen;

FIGS. 19-20 are side elevational views of the distal end of the medical catheter according to a second embodiment of the invention;

FIG. 21 is a perspective view of the distal end of the medical catheter;

FIG. 22 is a side elevational view of the catheter assemble for use in the diseased area of the blood vessel;

FIG. 23 is a plan view of the distal end of the medical catheter of FIG. 22;

FIG. 24 is a side elevational view of the catheter assemble for use in the diseased area of the blood vessel;

FIGS. 25-32 are plan views of the distal end of the medical catheter of FIG. 24;

FIGS. 33-34 are side elevational views of the distal end of the medical catheter according to a third embodiment of the invention;

FIG. 35 is a plan view taken along the line XXXV-XXXV of FIG. 34;

FIG. 36 is a side elevational view of the catheter assemble for use in the diseased area of the blood vessel;

FIGS. 37-38 are plan views of the distal end of the medical catheter of FIG. 36;

FIGS. 39-40 are side elevational views of the distal end of the medical catheter according to a fourth embodiment of the invention;

FIGS. 41-42 are plan views of the catheter assemble for use in the diseased area of the blood vessel;

FIG. 43 is a plan view of the distal end of the medical catheter of FIG. 42;

FIG. 44 is a plan view of the catheter assemble for use in the diseased area of the blood vessel;

FIGS. 45-46 are side elevational views of the medical catheter according to a fifth embodiment of the invention;

FIGS. 47-48 are side elevational views of the medical catheter on which a plurality of radiopaque films are provided according to a sixth embodiment of the invention;

FIG. 49 is a schematic view of a first and second rise-up portion of a second guide wire which is curvedly bent at inflection portions according to a seventh embodiment of the invention;

FIG. 50 is a schematic view showing a rotational area achieved by the first and second rise-up portion of the second guide wire;

FIG. 51 is a side elevational view of the catheter assemble for use in the diseased area of the blood vessel;

FIGS. 52-53 are plan views of the distal end of the medical catheter of FIG. 51;

FIG. 54 is a side elevational view of an elongate sheath according to an eighth embodiment of the invention;

FIG. 55 is a side elevational view of a guiding catheter;

FIG. 56 is a side elevational view of the rapid-exchange type catheter assemble;

FIG. 57 is a schematic view of therapeutical treatment with the use of the elongate sheath and the guiding catheter;

FIG. 58 is a schematic view of therapeutical treatment with the use of the medical catheter and the guiding catheter;

FIG. 59 is a side elevational view of the elongate sheath according to a ninth embodiment of the invention;

FIG. 60 is a side elevational view of the guiding catheter; and

FIG. 61 is a side elevational view of an over-the-wire type medical catheter assemble.

### DETAILED DESCRIPTION OF PREFERRED EMBODIMENTS OF THE INVENTION

In the following description of the depicted embodiments, the same reference numerals are used for features of the same type.

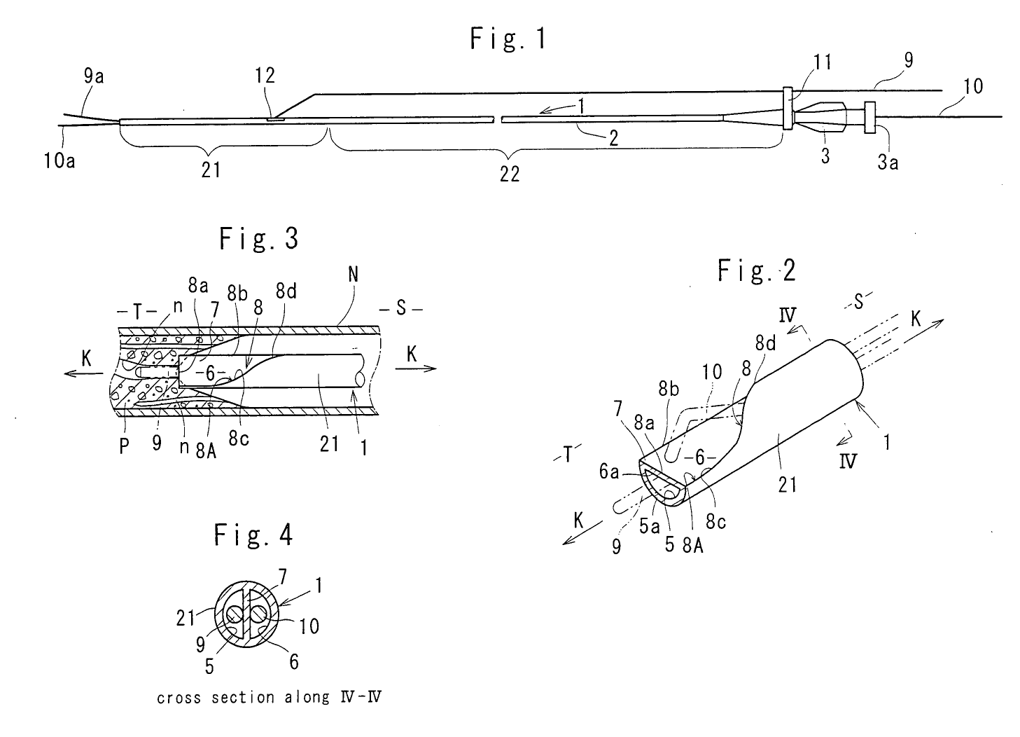

Referring to FIGS. 1 through 18, a medical catheter (shortened merely as “catheter” hereinafter) serves as a rapid-exchange type catheter assemble which enables users to a quick exchange of catheters according to a first embodiment of the invention. The catheter 1 forms a part of catheter assemble which has a first guide wire 9 and a second guide wire 10 to serve as a therapeutical treatment against a diseased portion P of the blood vessel N.

As shown in FIG. 1, the catheter 1 has an elongate tubular catheter body 2 and a connector 3 connected in series to a rear end portion which corresponds to a proximal end portion of the catheter body 2. The catheter body 2 measures approx. 1000-2000 (e.g., 1500 mm) in length and approx. 0.9-1.3 mm in diameter.

By way of illustration, the catheter body 2 has a front end portion 21 (approx. 170 mm in length) and the rest rear end portion 22, each formed by a synthetic resin representing a flexibilty and rigidity mixed in an appropriate combination.

As shown in FIGS. 2 and 4, the front end portion 21 has a circular cross section to form first and second lumens 5, 6 juxtaposed by separating an inner space with a common wall 7. The common wall 7 extends in a lengthwise direction K of the catheter 1 with the first and second lumens 5, 6 respectively formed at both sides of the catheter body 2. The first and second lumens 5, 6 have a D-shaped cross section respectively, and a circumferential length of the first lumen 5 is predetermined to be the same as that of the second lumen 6. Namely, the former angle (180 degrees) at the circumference is the same as the latter angle (180 degrees) at the circumference.

On the respective distal ends of the first and second lumen 5, 6, front openings 5a, 5b are defined respectively. The front opening 5a of the first lumen 5 has an open surface perpendicular to the lengthwise direction K.

As shown in FIG. 3, the front opening 6a of the second lumen 6 is defined on the outer surface of the catheter body 2 as a lateral slope opening 8 which inclines upward against the common wall 7 from a distal end T to a proximal end S of the catheter body 2. An inner circumferential edge 8A of the lateral slope opening 8 generally forms a curved right triangle by a distal edge 8a (distal end of the common wall 7), a lateral side edge 8b and a slantingly curved side edge 8c. The opening 8 directs the curved side edge 8c from one corner of the distal edge 8a toward the proximal end S while turning around the lengthwise direction K within 180 degrees (e.g., 180 degrees), and joins the lateral side edge 8b at an apex end 8d so as to reach the other corner of the distal edge 8a (closed loop). The opening 8 should be acceptable so long as the inner circumferential edge 8A inclines upward from the distal end T to the proximal end S while turning curved side edge 8c around the lengthwise direction K to form a closed loop as a whole.

Through the first lumen 5, the first guide wire 9 introduces its distal end 9a into the catheter body 2 via an insertion hole 12 to make the distal end 9a come out of the front opening 5a as shown in FIGS. 1, 5, 6.

Through the second lumen 6, the second guide wire 10 introduces its distal end 10a into the catheter body 2 via an access hole 3a of the connector 3 to make the distal end 10a come out of the lateral slope opening 8. Outer diameters of the first and second guide wires 9, 10 are predetermined to be 0.014 inches by way of example.

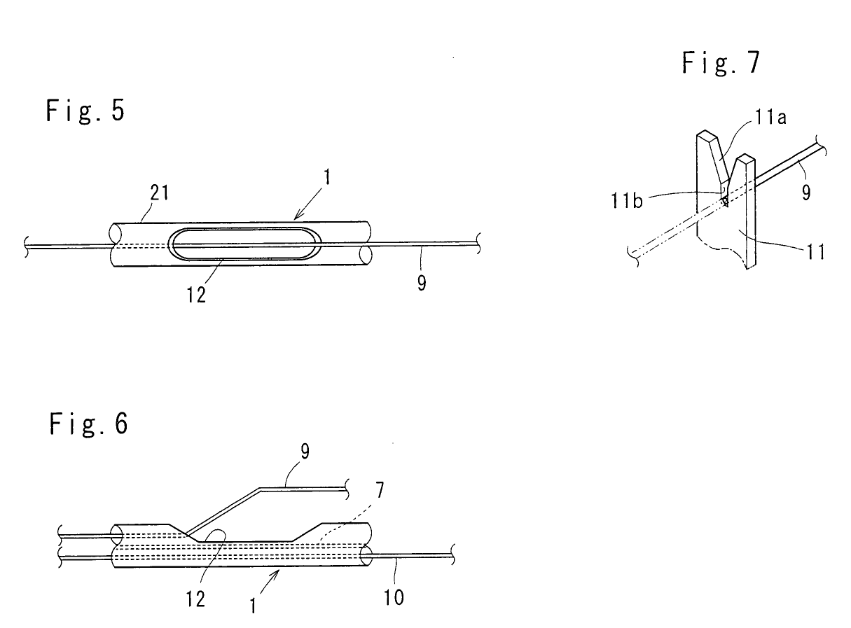

The insertion hole 12 is defined oblong on a middle of the catheter body 2 as shown in FIGS. 5, 6. An engagement tool 11 is mounted on the connector 3, and has a V-shaped notch 11a, an apex of which forms an engagement groove 11b to firmly receive the first guide wire 9 in an orthogonal direction to prevent the first guide wire 9 from coming off the engagement tool 11 as shown in FIG. 7.

Upon rotating the catheter 1 in the blood vessel N, the engagement tool 11 prevents the guide wires 9, 10 from being accidentally entangled each other.

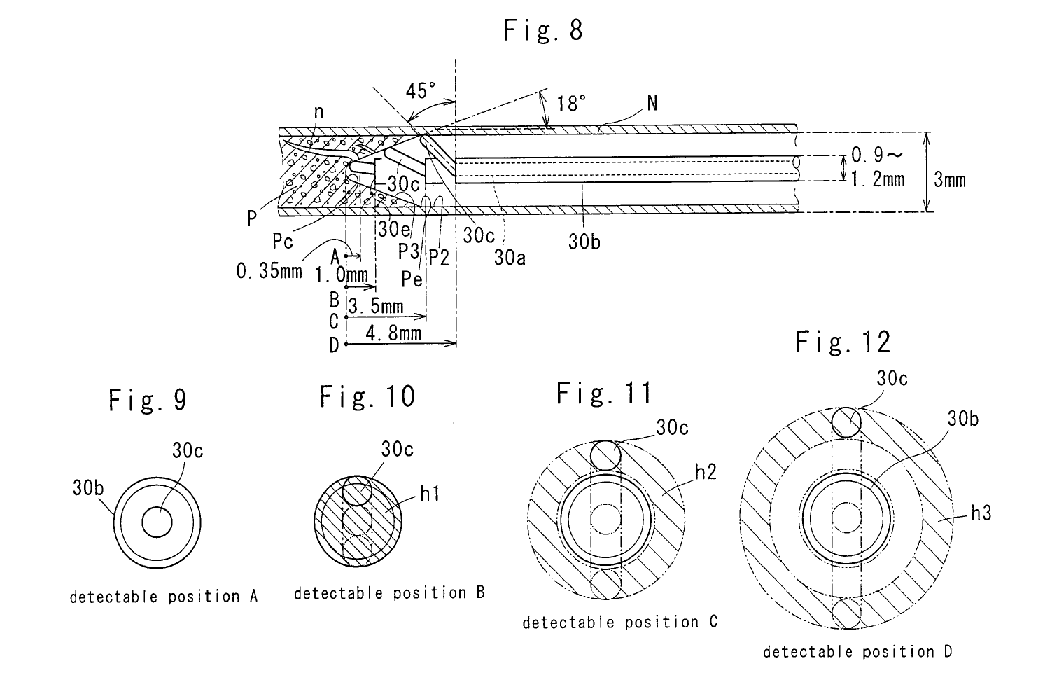

Since it is usual that a plurality of minute blood stream paths (n) develops in the occluded area of the blood vessel N, the first guide wire 9 makes the distal end 9adetect the minute blood stream paths (n) to perforate one of the minute blood stream paths (n) as shown in FIG. 8.

With the above structure described thus far, the catheter 1 has the first and second lumens 5, 6 to form a dual lumen catheter. The second guide wire 10 extends its distal end 10a beyond the lateral slope opening 8, it is possible for the distal end 10a to direct the movable or shiftable region in a predetermined three-dimensional way, thus extending a detectable region permitted for the distal end 10a to explore a perforative hole against minute blood stream paths (n) so as to make it easy to operationally lead the distal end 10a to the minute blood stream paths (n).

Namely, upon encountering the distal end 10a against the diseased area P (e.g., vascular stricture area or completely occluded area), it is possible for the operator to significantly extend the detectable region permitted for the distal end 10a to explore the minute blood stream paths (n), so as to enable the operator to easily perforate the diseased area P more than the conventional counterpart catheter could do.

FIG. 8 shows a comparative counterpart catheter 30b (0.9-1.2 mm in dia.), into which a guide wire 30a (0.3 mm in da.) is operationally inserted to detect a perforative position suitable for penetrating through a diseased area P3. When the guide wire 30a forms its distal end 30c straight, the perforative position happens to be only a place in which the guide wire 30a encounters the distal end 30 against a diseased surface P3 as shown in FIGS. 8, 9.

By way of illustration, a detectable position (A) in FIGS. 8, 9 is a place in which a top end 30e of the catheter 30b is away by 0.35 mm along the lengthwise direction K from a central cavity Pc of the diseased surface P3.

A detectable position (B) in FIG. 10 is a place in which the top end 30e of the catheter 30b is away by 1.0 mm along the lengthwise direction K from the central cavity Pc of the diseased surface P3. A detectable position (C) in FIG. 11 is an entrance Pe of the diseased surface P3 in which the top end 30e of the catheter 30b is away by 3.5 mm along the lengthwise direction K from the central cavity Pc of the diseased surface P3. A detectable position (D) in FIG. 12 is a place in which the top end 30e of the catheter 30b is away by 4.8 mm along the lengthwise direction K from the central cavity Pc of the diseased surface P3.

When the distal end 30c of the guide wire 30a is arcuately preformed by e.g., 45 degrees, the acuate distal end 30c enables the operator to shift the top end 30e via the detectable positions B, C to the detectable position D (normal vascular wall periphery P2 of the blood vessel N) upon therapeutically maneuvering the catheter 30b and the guide wire 30a in combination.

Consequently, the detectable position B permits the distal end 30c to move as indicated by a hatched circular area (h1) in FIG. 10. The detectable position C permits the distal end 30c to move as indicated by a hatched annular area (h2) in FIG. 11. The detectable position D permits the distal end 30c to move as indicated by a hatched annular area (h3) in FIG. 12.

During the process in which the guide wire 30a moves its top end 30e from the detectable position A to the detectable position D, the detectable region which the distal end 30c is permitted to explore the perforative hole comes to a total sum of the areas (h1), (h2) and (h3).

In this situation, upon curvedly bending the distal end 30c of the guide wire 30a, unless the catheter 30b is somewhat shifted together with the guide wire 30a toward the proximal end portion, it is not possible to extend the detectable region permitted for the distal end 30c to explore the perforative hole against the diseased surface P3.

At this moment, the catheter 30b must be held firmly to make the distal end 30c unstable against the diseased surface P3. In addition to the above, the patient is therapeutically treated in the pulsatory condition to make the distal end 30c more unstable, thus rendering it difficult for the operator to detect the minute blood stream paths (n) of the diseased surface P3.

By way of illustration, the guide wire 30a must be returned operationally by approx. 4.8 mm toward the proximal end portion in order to detect the normal vascular wall periphery P2 of the blood vessel N when the diseased surface P3 inclines by 18 degrees with the inner diameter of the blood vessel N designated as 3.0 mm in FIG. 8. At this location, the catheter 30b and the guide wire 30a are likely to be unstable with the patient treated in the pulsatory condition, and rendering it difficult to make the distal end 30c detect the perforative hole against the minute blood stream paths (n) of the diseased surface P3.

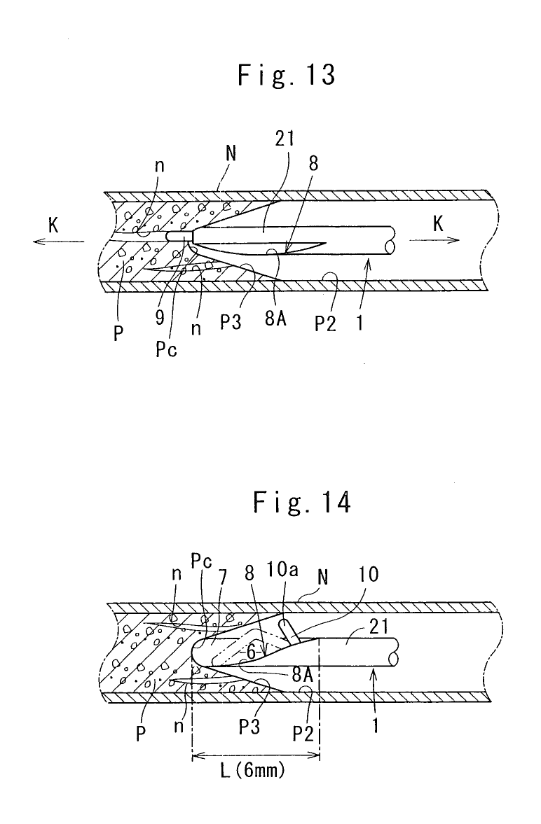

Contrary to the above comparative catheter 30b, according to the present invention as shown in FIGS. 13, 14, it is possible to make the distal end 10a of the second guide wire 10 detect the position suitable for the perforative hole with the top end of the catheter 1 engaged with the central cavity Pc of the diseased surface P3.

The following are procedures operationally needed to detect the position suitable for the perforative hole against the diseased surface P3.
(a) The catheter 1 is inserted into the blood vessel N to advance toward the diseased surface P3 with the first guide wire 9 set through the first lumen 5 as shown in FIG. 13.
(b) In this situation, the first guide wire 9 is operated to detect the position suitable for the perforative hole against the diseased surface P3 in the lengthwise direction K of the catheter 1. The catheter 1 supports a reactional force developed when pushing the first guide wire 9 against the diseased surface P3, and making it easy to operationally advance the first guide wire 9 forward against the diseased surface P3.
(c) When the operator finds it difficult for the first guide wire 9 to detect the position suitable for the perforative hole against the diseased surface P3 in the lengthwise direction K of the catheter 1, the second guide wire 10 is inserted into the second lumen 6 of the catheter 1 with the distal end preformed into e.g., a dog-legged configuration as shown by a phantom line in FIG. 14.
(d) The second guide wire is operated so that the distal end 10a moves forward and rearward along the inner circumferential edge 8A of the lateral slope opening 8 with the top end of the catheter 1 engaged with the diseased surface P3. This enables the operator to extend the detectable position from the central cavity Pc to the normal vascular wall periphery P2 of the blood vessel N.

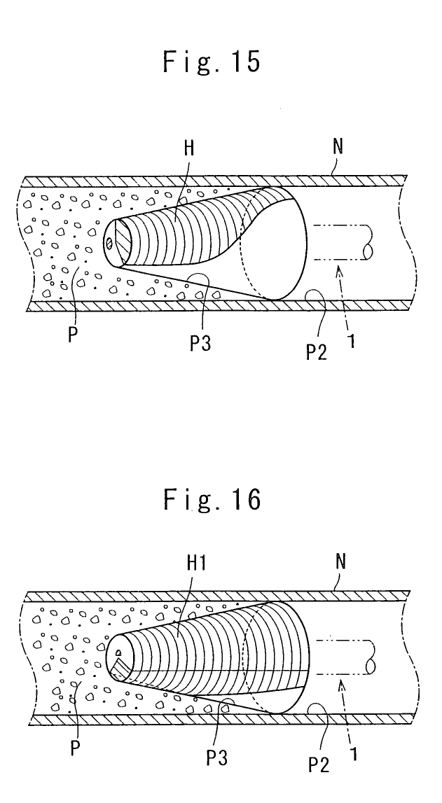

In fact, it is possible to make the second guide wire 10 detect up to the normal vascular wall periphery P2 by determining the length L of the lateral slope opening 8 to be e.g., 6.0 mm along the lengthwise direction K when the diseased condition of the blood vessel N is the same as that observed in FIG. 8.
(e) The second guide wire 10 is operationally rotated slightly around its axis together with the catheter 1 while holding the top end of the catheter 1 to engage with the diseased surface P3. At the slightly rotated position, the first guide wire 9 is operated to explore the diseased surface P3 along the lengthwise direction K, and the second guide wire 10 is operated to detect the position suitable for the perforative hole against an inner wall of the diseased surface P3 of the blood vessel N.
(f) By repeating the above procedures, it becomes possible to explore an entire extent of the diseased portion P such as the vascular stricture area or completely occluded area of the blood vessel N along their circumferential direction. By way of example, FIG. 15 positionally shows the catheter 1 before it is rotated in the blood vessel N, and FIG. 16 shows the catheter 1 after it is rotated by 90 degrees from the position of FIG. 15 in the blood vessel N.

As a consequence, the second guide wire 10 enables the operator to extend the detectable region in the three-dimensional way as indicated at a hatched area H in FIG. 15 and a hatched area H1 FIG. 16 (H+H1) upon exploring the position suitable for the perforative hole.
(g) The front opening 5a may be defined as a gradient opening rising upward from the distal end portion T to the proximal end portion S as described in detail hereinafter (second embodiment of the invention). The gradient opening has a front edge and a rear edge, both of which reside on a common plane, and having a maximum major length in the gradient direction. In this situation, as shown at a hatched area H2 in FIG. 18, it is possible to extend the detectable region for the perforative hole against the diseased surface P3 by moving the first guide wire 9 along an inner circumferential edge of the gradient opening.

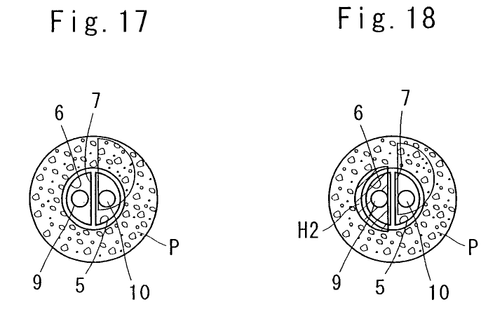

FIG. 17 is a plan view of the top end of the catheter 1 (FIG. 14) shown for the purpose of indicating the detectable region for the perforative hole. FIG. 18 is a plan view showing the gradient opening defined on the distal end of the first lumen 5.

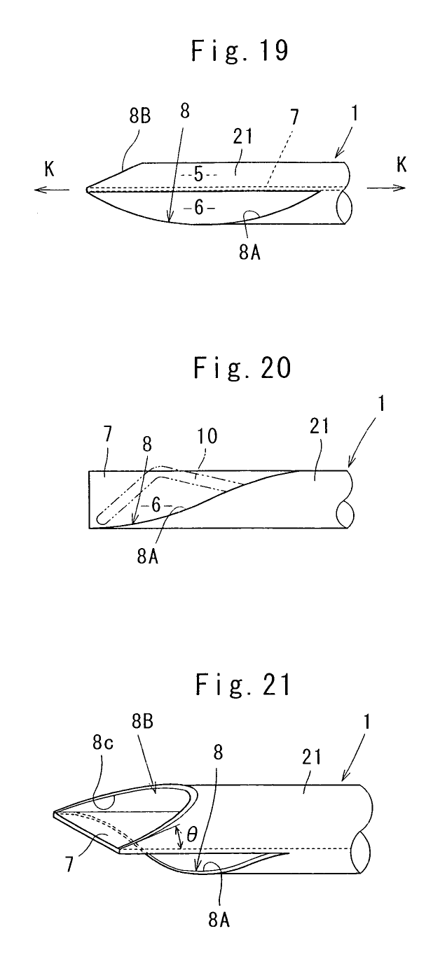

FIGS. 19 through 32 show a second embodiment of the invention in which the gradient opening 8B is defined instead of the front opening 5a, an open surface of which is perpendicular to the lengthwise direction K. As shown in FIGS. 19, 20, 21, the gradient opening 8B has the front edge and rear edge, both of which reside on the common plane, and having the maximum major length along a central extension in the gradient direction. The gradient opening 8B has the open surface forming an angle (θ) of e.g., 15-45 degrees against common wall 7 as shown in FIG. 21. It is possible to extend the detectable region for the perforative hole against the diseased surface P3 by moving the first guide wire 9 along an inner circumferential edge of the gradient opening 8B as already shown at the hatched area H2 in FIG. 18.

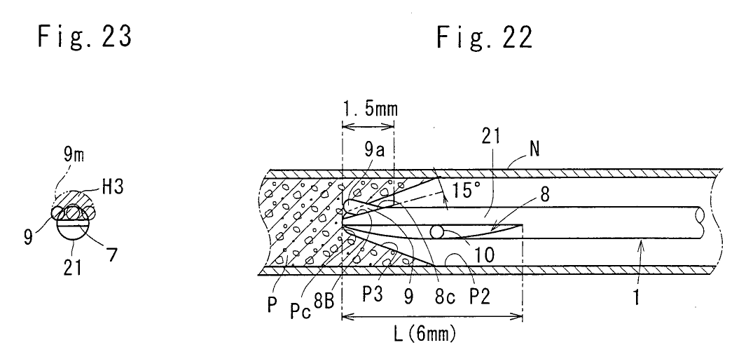

upon moving the distal end 9a of the first guide wire 9 forward and rearward along the inner circumferential edge of the gradient opening 8B as shown in FIGS. 21, 22, the movement of the distal end 9a forms an envelope 9m which is projected on a plane to represent an hatched area H3 in FIG. 23 as the detectable area for the perforative hole.

With the gradient opening 8B on the distal end of the first lumen 5, the hatched area H3 forms generally semi-circular with an increased diameter. This means to extend the detectable region permitted for the first guide wire 9 to move toward the diseased surface P3 (FIG. 18).

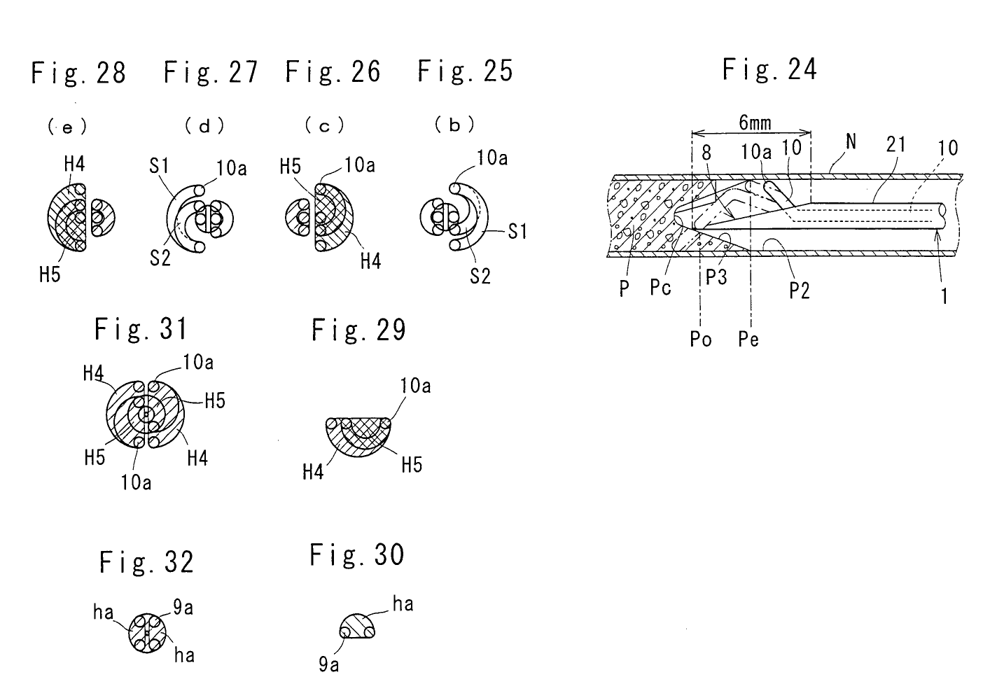

FIGS. 24 through 32 show a detectable action permitted for the second guide wire 10 in the second lumen 6, together with a detectable action permitted for the first guide wire 9 in the first lumen 5.

Upon moving the distal end 10a of the second guide wire 10 to the entrance Pe of the diseased surface P3 (FIG. 24), a maximum band area S1 is achieved as a planar region which is permitted for the distal end 10a of the second guide wire 10 to explore the perforative hole at the entrance Pe in accompany with the rotational operation of the catheter as shown in FIG. 25.

Upon moving the distal end 10a of the second guide wire 10 to the occluded depth area Po of the diseased surface P3, a maximum band area S2 is achieved as a planar region which is permitted for the distal end 10a of the second guide wire 10 to explore the perforative hole at the occluded depth area Po in accompany with the rotational operation of the catheter as shown in FIG. 25.

When moving the first guide wire 9 back and forth in the lengthwise direction K along the inner circumferential edge of the lateral slope opening 8, a detectable region for the perforative hole against the diseased surface P3 is obtained as an integrated sphere of an inner section H4 within the maximum band area S1 and an inner section H5 within the maximum band area S2 (FIG. 26).

FIG. 27 shows the maximum band areas S1, S2 among the detectable region permitted for the first guide wire 9 to explore the perforative hole when the catheter 1 is rotated by 180 degrees from the condition of FIG. 25. FIG. 28 shows the detectable region when the catheter 1 is rotated by 180 degrees from the condition of FIG. 26. FIG. 29 shows the detectable region when the catheter 1 is rotated by 90 degrees from the condition of FIG. 26.

FIG. 31 shows a total sum of the detectable region in FIG. 26 and the detectable region in FIG. 28. That is an entire detectable region permitted for the first guide wire 9 to explore the perforative hole when operating the first guide wire 9 along an entire circumferential inner wall of the diseased surface P3 from the entrance Pe to the occluded depth area Po. FIG. 30 is identical to FIG. 23, and showing the detectable region (denoted by ha) permitted for the distal end 9a to move in the lateral slope opening 8B to explore the perforative hole against the diseased surface P3.

FIG. 32 shows the detectable region permitted for the first guide wire 9 to move in the lateral slope opening 8B to explore the entire circumferential inner wall of the diseased surface P3. Since the detectable region of the first guide wire 9 in the lateral slope opening 8B is described in FIGS. 22, 23, the detectable region of the first guide wire 9 in the lateral slope opening 8B of FIGS. 25, 26, 27, 28 and 30 is omitted.

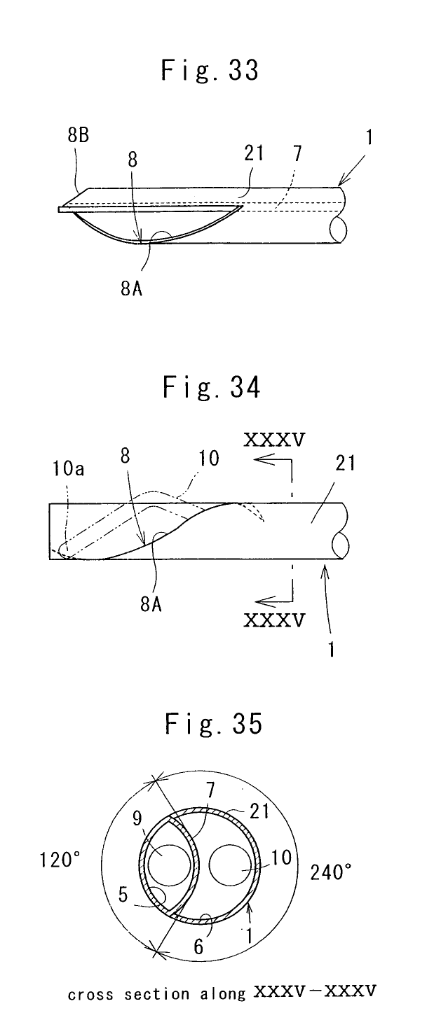

FIGS. 33 through 38 show a third embodiment of the invention in which the outer circumferential length of the second lumen 6 is predetermined to be greater than that of the first lumen 5. The second lumen 6 has a crescent-shaped cross section, while the first lumen 5 has a spindle-shaped cross section so that the former' s cross sectional area is greater than the latter's cross sectional area.

An outer circumferential arc portion of the first lumen 5 partly overlaps that of the second lumen 6 in order to define an arcuately convex-shaped common wall 7 as shown in FIGS. 33, 35. Although the arcuately convex-shaped common wall is structurally different from the common wall 7 of the first embodiment of the invention, the same reference numeral 7 is used to the arcuately convex-shaped common wall for the purpose of convenience.

By way of example, the outer circumferential arc portion of the first lumen 5 has 120 degrees, and that of the second lumen 6 has 240 degrees as angles at their circumferences as shown in FIG. 35.

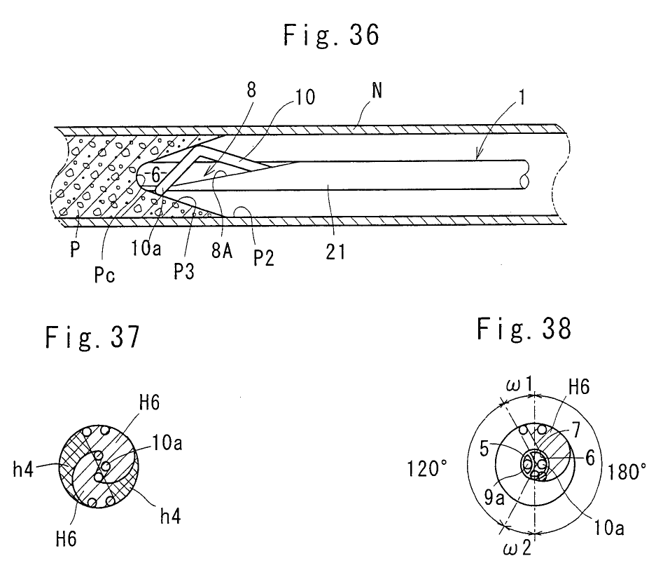

Upon therapeutically treating the diseased area P as shown in FIG. 36, with the first and second guide wires 9, 10 set in the first and second lumens 5, 6 respectively, the catheter 1 is inserted into the blood vessel N in order to detect the position suitable for exploring the perforative hole against the diseased surface P3 in the same procedures as described in the first embodiment of the invention.

With the circumferential arc portion of the second lumen 6 having 240 degrees as the angle at the circumference, the angle at the circumference becomes greater by 60 degrees {(ω1+ω2) in FIG. 38} than that (180 degrees) of the first embodiment of the invention. This enables the operator to extend the detectable region permitted for the second guide wire 10 to explore the diseased surface P3 as shown at a hatched area H6 in FIG. 37.

With some rotational operation of the catheter 1, it is possible to add a detectable increment region as shown at a hatched area h4 in FIG. 37. This enables the operator to explore the position suitable for the second guide wire 10 to detect the perforative hole against the diseased surface P3 along an entire inner circumferential direction of the vascular wall.

It is to be noted that the hatched area H6 of FIGS. 37, 38 means an integrated set of probing points in which the second guide wire 10 renders the distal end 10a engageable with the diseased surface P3 upon detecting the perforative hole against the diseased surface P3.

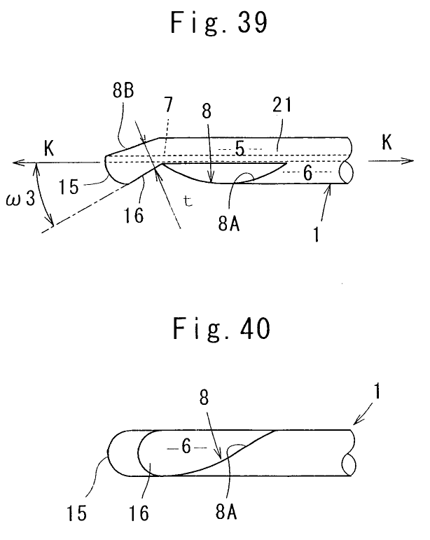

FIGS. 39, 41 through 44 show a fourth embodiment of the invention in which a semi-spherical head portion 15 is provided on a distal end of the catheter 1 at the side of the second lumen 6 as shown in FIGS. 39, 40. The head portion 15 has a central convex portion and having an underside slope surface 16 extending from the distal end to the common wall 7 at an obtuse angle ω3 formed against the lengthwise direction K. As a result, a thickness (t) of the head portion 15 progressively decreases toward the common wall 7.

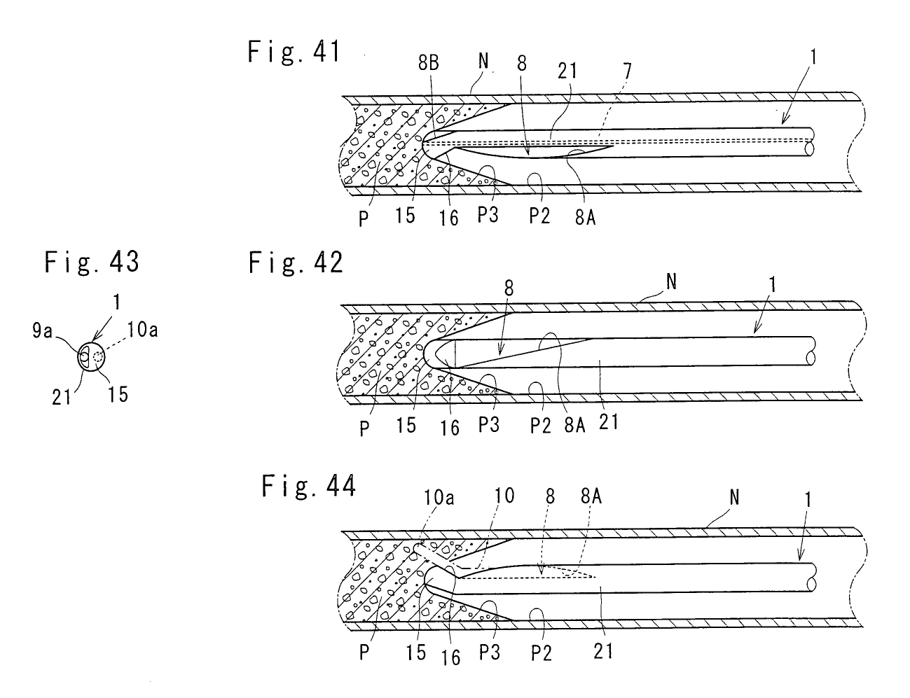

When the catheter is operationally rotated with the head portion 15 engaged with the diseased surface P3 upon detecting the position suitable for the perforative hole against the diseased surface P3 as shown in FIGS. 41, 42, 43, the semi-spherical head portion 15 enables the operator to readily rotate the catheter 1 so as to facilitate a smooth rotational operation of the catheter 1 as shown in FIG. 44.

With the slope surface 16 inclined toward the common wall 7, the slope surface 16 supports a pushing force of the second guide wire 10 against the diseased surface P3 to help the second guide wire 10 penetrate into the diseased surface P3. It is to be noted that instead of the semi-spherical configuration, a shape of the head portion 15 may be a cylindrical or bullet-shaped configuration, otherwise it may be a conical body such as, for example, ellipsoid or hyperboloid.

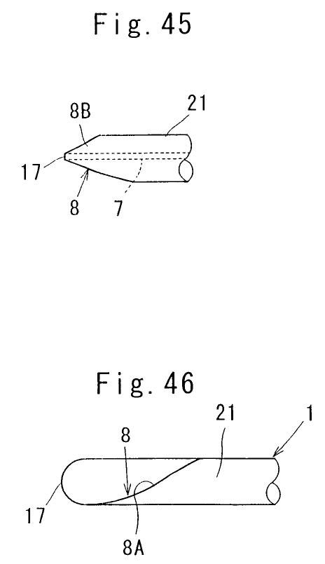

FIGS. 45, 46 show a fifth embodiment of the invention in which a distal end of the common wall 7 has a semi-circular configuration together with the distal ends of the first and second lumens 5, 6.

With the distal end of the common wall 7 formed semi-circular in configuration, it is possible to readily rotate the catheter 1 so as to facilitate a smooth rotational operation of the catheter 1 as mentioned in the fourth embodiment of the invention.

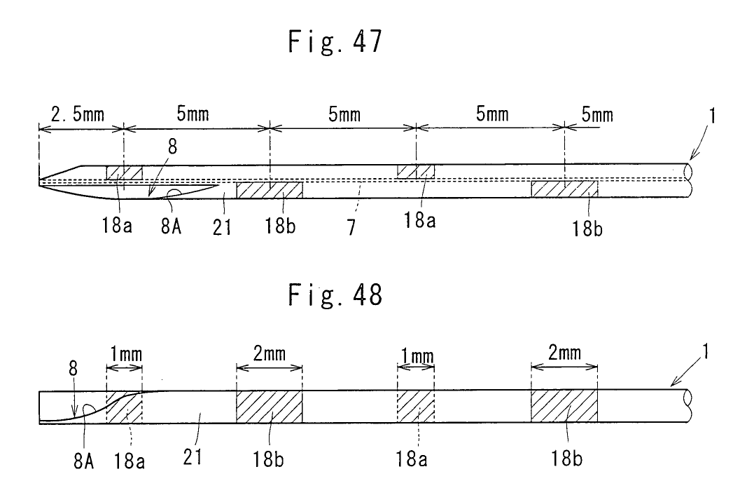

FIGS. 47, 48 show a sixth embodiment of the invention in which a series of semi-cylindrical radiopaque films 18a, 18b (silver or platinum film) is provided on a distal end portion of the catheter 1.

By way of example, the film 18a measures 1.0 mm in width and attached to an outer surface of the first lumen 5. The film 18b measures 2.0 mm in width and attached to an outer surface of the second lumen 6. These films 18a, 18b are aligned alternately at regular intervals (e.g., 5.0 mm) in the lengthwise direction K, and orthogonally correspond each other with the common wall 7 interposed.

With the radiopaque films 18a, 18b served as markers, it is possible to observe the widths of the films 18a, 18b on a fluoroscopic image screen (not shown) so as to distinguish the first lumen 5 from the second lumen 6, while at the same time, visually recognizing the first guide wire 9 and the second guide wire individually.

By consecutively arranging the films 18a, 18b in combination at the regular intervals (5.0 mm), it is possible to achieve a size measurement function against the diseased portion P. It is to be noted that the films 18a, 18b may be different in thickness to make a luminous distinction upon recognizing the films 18a, 18b on the fluoroscopic image screen.

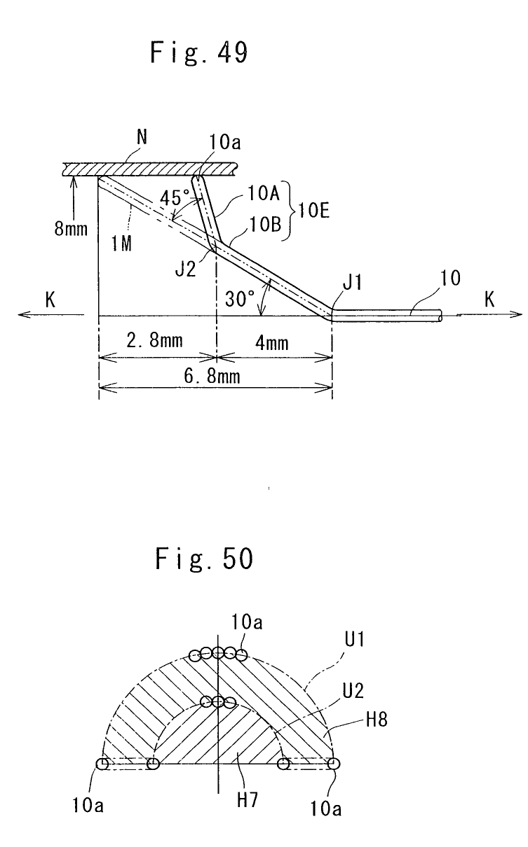

FIGS. 49 through 53 show a seventh embodiment of the invention in which the second guide wire 10 forms two inflection portions J1, J2 at the distal end 10a.

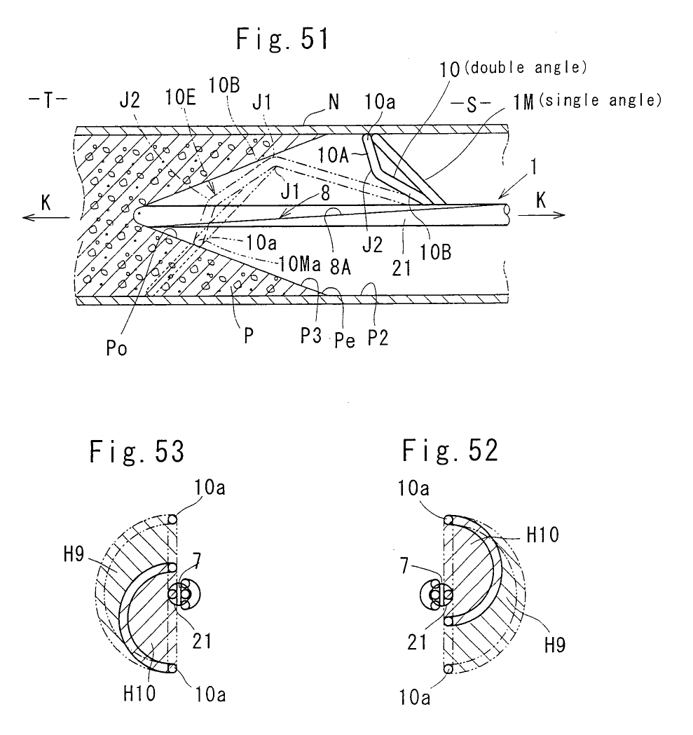

The second guide wire 10 engages the inflection portions J2, J1 with the diseased surface P3, so that the inflection portions J2, J1 function as supports of a reaction against the pushing force from the second guide wire 10, so as to impart a forward propelling force to the second guide wire 10 when the diseased surface P3 has an inner wall progressively decreasing its diameter more as approaching forward as shown in FIG. 51.

In the second lumen 6, the second guide wire 10 has a distal end portion 10E divided into a first rise-up section 10A and a second rise-up section 10B by the inflection portions J2, J1, so as to generally form an arcuately bent or sinuously curved configuration as shown in FIG. 49.

By way of illustration, the first rise-up section 10A acts as a first angle portion to form 45 degrees against the second rise-up section 10B while the second rise-up section 10B acts as a second angle portion to form 30 degrees against the lengthwise direction K. A total angle of the first rise-up section 10A and a second rise-up section 10B is predetermined to be less than 90 degrees as attained by calculatedly adding 45 degrees to 30 degrees. The reason why the total angle is predetermined to be less than 90 degrees, is that a rotational operation and penetrability of the second guide wire 10 against the diseased surface P3 deteriorate when the total angle exceeds 90 degrees.

When rotating the distal end portion 10E around the inflection portion J1 at the proximal end of the second catheter 10 in the blood vessel N (8.0 mm in dia.) as shown in FIG. 49, the distal end 10a of the first rise-up section 10A draws a locus as shown by an outer loop fringe U1 in FIG. 50.

When pulling the second guide wire 10 rearward from the above position while rotating the second guide wire 10 until the inflection portion J2 changes to the inflection portion J1, the distal end 10a draws a locus from the outer loop fringe U1 to an inner loop fringe U2 in FIG. 50. The resultant locus region is a semi-annular rotary area H8 surrounded by the outer loop fringe U1 and the inner loop fringe U2 in FIG. 50.

When further pulling the second guide wire 10 from the above position, the distal end 10a draws a semi-circular rotary area H7 with the inner loop fringe U2 as an outer circumference during process in which the distal end portion 10E is retracted into the second lumen 6.

During the process in which the distal end portion 10E is pulled while the second guide wire 10 is rotated, the distal end 10a of the first rise-up section 10A resultantly draws a total region extending from the semi-annular rotary area H8 to the semi-circular rotary area H7.

When comparing the second guide wire 10 (double angle type) to a guide wire 1M (single angle type) in which only one inflection portion J1 is provided on the distal end portion 10E, the distal end portion of the second guide wire 10 requires 4.0 mm in the lengthwise direction K, as opposed to the guide wire 1M in which the distal end portion requires 6.8 mm in the lengthwise direction K with an inner diameter of the blood vessel N as 8.0 mm.

The inflection portions J1, J2 make the distal end portion 10E dimensionally reduce by 2.8 mm (approx. 41%) in the lengthwise direction K, thus making it possible to extend the detectable region against the diseased surface P3 in the diametrical direction while reducing the distance in the lengthwise direction K.

The dimensional reduction of the distal end portion 10E is especially favorable upon therapeutically treating the diseased portion P of the iliac artery, an inner diameter of which tends to increase. It is to be noted that instead of the two inflection portions J1, J2, three or more inflection portions may be provided. The inflection portions may be provided on the first guide wire 9 in addition to the second guide wire 10. Otherwise, the inflection portions may be provided only on the first guide wire 9 without providing it on the second guide wire 9.

When inserting the second guide wire 10 and the catheter 1 into the blood vessel N as a catheter assemble in the manner as shown in FIG. 51, a hatched area H9 in FIG. 52 represents the detectable region permitted for the second guide wire 10 to explore the diseased surface P3 at the entrance Pe.

A hatched area H10 in FIG. 52 represents the detectable region permitted for the second guide wire 10 to explore the diseased surface P3 at the occluded depth area Po. When the catheter assemble is rotated by 180 degrees in the clockwise direction from the position of FIG. 52, the detectable regions H9, H10 are rotated by 180 degrees in the same direction as shown in FIG. 53.

As shown in FIG. 51, the second guide wire 10 serves as the double angle type because the second guide wire 10 has the inflection portions J1, J2. The guide wire 1M serves as the single angle type because the guide wire IM has the single inflection portion J1. As shown by a phantom line in FIG. 51, the second guide wire 10 comes out of the catheter 1 to advance while being bent at the inflection portions J1, J2 in a double refractive configuration to reach the distal end 10 at the diseased surface P3. The guide wire 1M places its distal end 10Ma at the same detectable position as the guide wire 10 places its distal end 10a. Because the inflection portion J1 engages with the vascular wall, a vascular engagement force developed as a reaction against the vascular wall transforms the engagement force such a direction as to permit an easy penetration into the minutes blood stream paths (n) of the diseased surface P3 upon advancing the second guide wire 10 (guide wire 1 M) toward the diseased surface P3.

Similarly, the inflection portion J2 of the second guide wire 10 is used to permit an easy penetration deep into the minute blood stream paths (n) in an orientation perpendicular to the lengthwise direction of the blood vessel N. In this way, the second guide wire 10 engages the inflection portions J1, J2 against the vascular wall, and transforms the engagement force and direction toward the easy penetration into the minute blood stream paths (n).

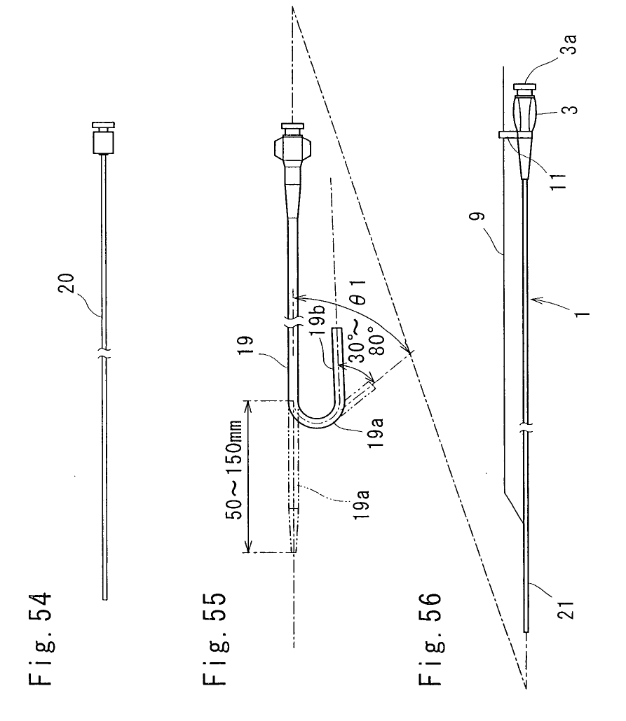

FIGS. 54 through 57 show an eighth embodiment of the invention in which a catheter assemble is provided by combining the catheter 1 having the first guide wire 9 and the second guide wire 10 with a guiding catheter 19 and an elongate sheath 20 as shown in FIGS. 54, 55, 56.

As shown in FIG. 55, the guiding catheter 19 has a U-shaped bend portion 19a arcuately preformed by approx. 180 degrees at the proximal side (approx. 50-150 mm) from an distal end of the guiding catheter 19. It is to be noted that the angle of the U-shaped bend portion 19a is not necessarily confined to 180 degrees, any angle may be acceptable so long as the angle is within 130-230 degrees.

Upon inserting the elongate sheath 20 into the guiding catheter 19, the elongate sheath 20 stretches the U-shaped bend portion 19a straight as shown by a phantom line in FIG. 55.

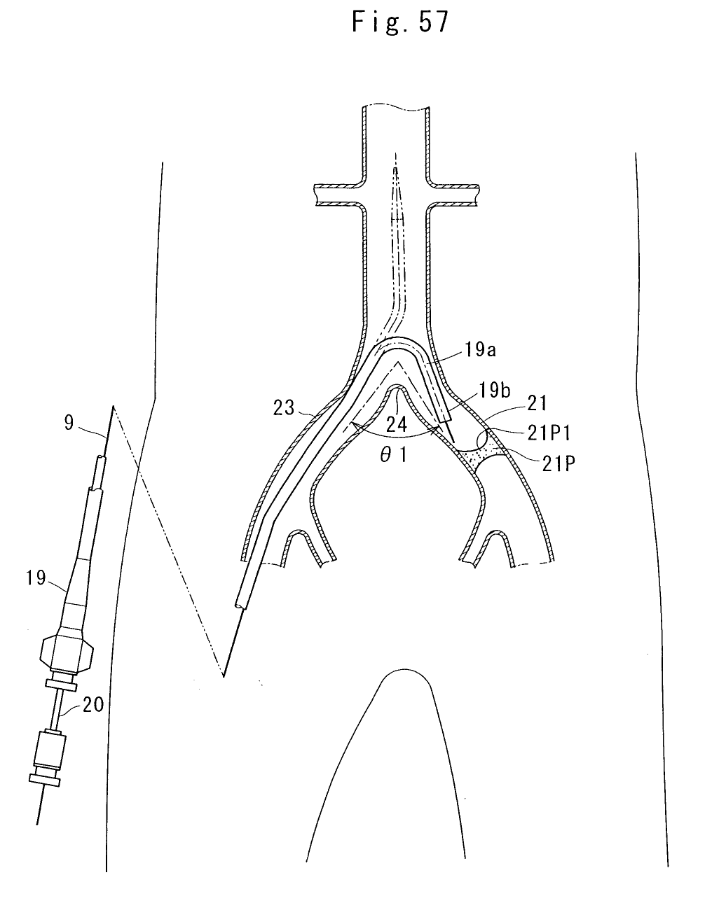

In order to meet a lateral side portion 19b of the U-shaped bend portion 19 with a bifurcated portion 24 of an iliac artery (FIG. 57), the lateral side portion 19b of the U-shaped bend portion 19a is beforehand bent 30-80 degrees (50±30 degrees) more than a bifurcated angle θ1 of the iliac artery by considering a plastic deformation and a spring back of the U-shaped bend portion 19a upon pulling the elongate sheath 20 out of the guiding catheter 19.

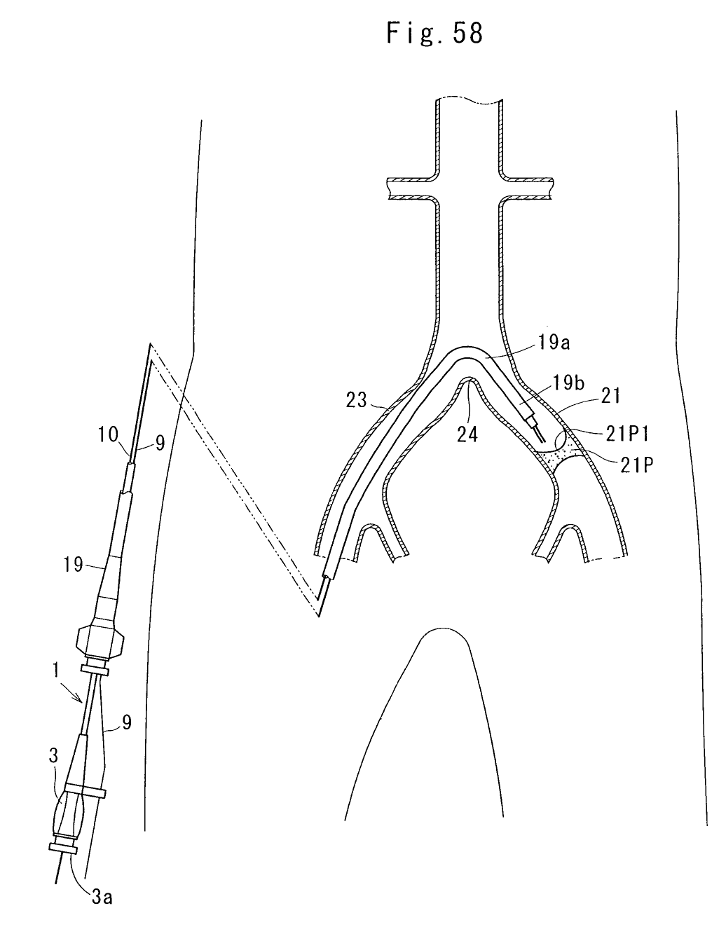

Following are procedures necessary to introduce the catheter 1 into a diseased portion 21P of the heterolateral iliac artery.
(a) Due to an insertion of the elongate sheath 20 into the guiding catheter 19, the elongate sheath 20 stretches the U-shaped bend portion 19a straight. Thereafter, the first guide wire 9 is insertionally set within the elongate sheath 20 as shown in FIGS. 56, 57.
(b) With the elongate sheath 20 set in the guiding catheter 20, the guiding catheter 20 is percutaneously inserted into the ortholateral iliac artery 23 to reach the bifurcated portion 24 of the iliac artery. In this instance, the first guide wire 9 preferentially puts an entry of its distal end into the heterolateral iliac artery 26.
(c) The guiding catheter 19 is adjusted at its U-shaped bend portion 19a to shift along the bifurcated portion 24 of the iliac artery while gradually pulling the elongate sheath 20 back to the proximal side. In so doing, the guiding catheter 19 changes its distal end to direct toward the heterolateral iliac artery 26, and introduce the distal end into the heterolateral iliac artery 26.
(d) After pulling the elongate sheath 20 out of the guiding catheter 19, the catheter 1 is insertionally set in the guiding catheter 19 as shown in FIG. 58.

In the case of the rapid-exchange type catheter assemble, the first lumen 5 is inserted to a rear end of the first guide wire 9 to insertionally introduce the first guide wire 9 upon insertionally setting the catheter 1 in the guiding catheter 19.
(e) Then the catheter 1 together with the first guide wire 9 is introduced into the diseased portion 21P of the heterolateral iliac artery so as to explore the position suitable for advancing the distal end toward the perforative hole of the diseased surface P3. If the suitable position is not detected, in order to extend the detectable region, the second guide wire 10 is inserted into the second lumen 6 to extensively detect the suitable position at the diseased portion 21P of the heterolateral iliac artery.

With the use of the catheter assemble having the catheter 1, the elongate sheath 20 and the guiding catheter 19, the following advantages are obtained.
(a) Upon inserting a guiding catheter 19 into a bifurcated portion 24 of the iliac artery to therapeutically treat the diseased area of the iliac artery, because the bifurcated portion 24 forms a steep angular portion as a chevron-shaped configuration, it is necessary for the operator to sharply turn the catheter assemble by more than 130 degrees against the direction in which the catheter assemble is inserted. For this reason, it is by no means easy to insert a straight tube portion of the catheter assemble into the bifurcated portion 24 of the iliac artery.

When the distal end portion of the guiding catheter is arcuately bent for therapeutically treating the coronary artery, and the distal end portion is generally subjected to an operational reaction on the way to advance over the bifurcated portion of the iliac artery because of a limited length of the distal end portion retained in the heterolateral iliac artery, the operational reaction would float the guiding catheter (approx. 2.0 mm in dia.) off the bifurcated portion of the iliac artery (approx. 20.0 mm in dia.)
(b) Contrary to the straight tube portion of the above guiding catheter, the guiding catheter 19, according to the invention, has the arcuately U-shaped bend portion 19a at a proximal portion away by approx. 50-150 mm from the distal end of the guiding catheter 19. It is possible to insure a sufficient length of the U-shaped bend portion 19a at the heterolateral iliac artery, thus preventing the guiding catheter 19 from coming off the bifurcated portion 24 of the iliac artery without floating in the blood streams, so as to attain the therapeutical improvement against the diseased portion P.
(c) The guiding catheter 19 is provided because it is not only the catheter 1 which is subjected to the reactional force from the first guide wire 9 and the second guide wire 10, and the provision of the guiding catheter 19 is to counteract an even more reinforced reactional force. The reactional force resultantly produces a forward propelling force in an increased magnitude.
(d) The catheter 1 is used because an inner diameter of the catheter 1 is approx. 0.9-1.2 mm, and thereby enabling the operator to insert the catheter 1 into the diseased surface 21P1 of the narrow heterolateral iliac.

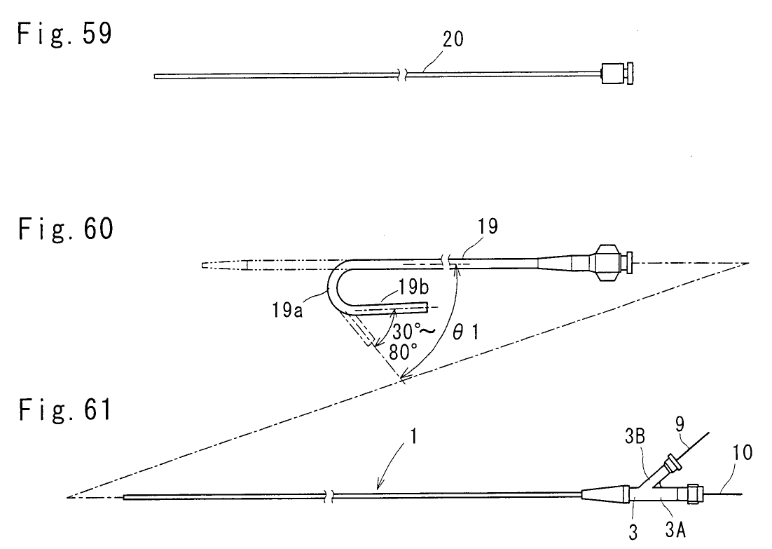

FIGS. 59 through 61 show a ninth embodiment of the invention in which an over-the-wire type catheter assemble is used instead of the rapid-exchange type catheter assemble.

The over-the-wire type catheter assemble has the common guiding catheter 19 and elongate sheath 20 with the rapid-exchange type catheter assemble. The connector 3 is divided into a main tube 3A and a diverted tube 3B, and the first guide wire 9 is inserted into the main tube 3A to pass through the first lumen 5 while the second guide wire 10 is inserted into the diverted tube 3B to pass through the second lumen 6.

### Modification Forms

(a) The first guide wire 9 is inserted into the first lumen 5 while the second guide wire 10 is inserted into the second lumen 6, however, the first guide wire 9 may be inserted into the second lumen 6 while the second guide wire 10 is inserted into the first lumen 5.

It is preferable to select one lumen which becomes to be straight with a lesser bending angle among the first lumen 5 and the second lumen 6 upon extending the detectable region permitted for the first or second guide wire to explore the perforative hole against the diseased surface P3.
(b) The first guide wire 9 and the second guide wire 10 may be formed by an elongate member and a helical spring body connected to the distal end of an elongate member, or an elongate member, an outer surface of which a synthetic film is coated on. The helical spring body is made of a stainless steel or a Ni-Ti based alloy, and the synthetic film is formed by a polyamide or fluoro-based resin.
(c) The catheter body may be made of a synthetic straight tube flexible in front and rigid in rear, or a synthetic straight tube. An outer surface of the synthetic straight tube is covered with a mesh-work metal braid, on an outer surface of which a synthetic resin layer is coated. Alternatively, the catheter body may be made of a synthetic straight tube, around an outer surface of which a single wire or a multitude of wires are helically wound. The synthetic resin layer is formed by a polyamide or fluoroplastics. The helical spring body is made of a stainless steel or a Ni-Ti based alloy. Otherwise, the helical spring body may be formed by connecting a stainless steel wire to a Ni-Ti alloyed wire in an appropriate combination.
(d) The elongate sheath 20 may be formed to be tapered off by a polyamide tube (synthetic tube).
(e) As the case of the catheter 1, the guiding catheter 19 may be a synthetic straight tube, on an outer surface of which a mesh-work metallic braid is provided and further coated with a synthetic resin layer. The mesh-work metallic braid may be omitted at the U-shaped bend portion 19a which is preformed from the distal end shortly toward the proximal side of the guiding catheter 19 (intermittent braid structure).
(f) As for the catheter 1, the guiding catheter 19, the first guide wire 9 and the second guide wire 10, on outer surfaces of the above members, a hydrophilic polymer (e. g., polyvinylpyrrolidone) may be coated to exhibit a lubricous property with a good insertability when moistened.

What is claimed is:

1. A medical catheter having a dual lumen each partitioned by a common wall, a distal end of said dual lumen having an opening area, through which a guide wire is introduced to pass;
wherein at least one of said opening areas of said dual lumen inclines as a lateral slope opening along said common wall from a distal portion to a proximal portion of said dual lumen.

2. The medical catheter according to claim 1, wherein a catheter body has a circular cross section, and each of said dual lumen has a D-shaped cross section and the same outer circumferential length.

3. The medical catheter according to claim 1, wherein one of said dual lumen has an outer circumferential length greater than that of the rest of said dual lumen, said lateral slope opening residing on said lumen, an outer circumferential length of which is greater than that of the rest of said dual lumen.

4. The medical catheter according to claim 1, wherein a distal end of said common wall is formed to be arcuately convex-shaped configuration.

5. The medical catheter according to claim 1, wherein said lumen which has said lateral slope opening, has a curved surface head portion having a cylindrical slope surface, semi-bullet surface or semi-spherical surface at a distal edge of said lateral slope opening.

6. A catheter assemble of said medical catheter according to claim 1 and guide wires inserted into said dual lumen, said guide wires being in the form of a curved or arcuate configuration and having one, two or three inflection portions with a total bending angle defined within 90 degrees.

7. A catheter assemble according to claim 6, wherein a guiding catheter is provided which has an arcuately bent portion having an angular range of 130-230 degrees at a remote portion away by 50-150 mm from a distal end of said guiding catheter, and an elongate sheath is inserted into said arcuately bent portion so as to stretch said arcuately bent portion with said guide wire inserted into said elongate sheath.
